(19) United States
(12) Patent Application Publication
(10) Pub. No.: US 2018/0353195 A1
(43) Pub. Date: Dec. 13, 2018

(54) MEDICAL DEVICES, KITS, AND METHODS FOR STONE EXTRACTION

(71) Applicant: Cook Medical Technologies LLC, Bloomington, IN (US)

(72) Inventors: John C. Sigmon, JR., Winston-Salem, NC (US); Shaun D. Gittard, Winston-Salem, NC (US); Gregory Hardy, Winston-Salem, NC (US)

(73) Assignee: Cook Medical Technologies LLC, Bloomington, IN (US)

(21) Appl. No.: 16/004,973

(22) Filed: Jun. 11, 2018

Related U.S. Application Data
(60) Provisional application No. 62/518,900, filed on Jun. 13, 2017.

(51) Int. Cl. A61B 17/221; A61B 17/22; A61B 17/225

(52) U.S. Cl. CPC A61B 17/221; A61B 17/22012; A61B 18/26; A61B 2017/2253; A61B 17/2251

(57) ABSTRACT

Methods, medical devices, and kits, and more particularly methods, devices, and kits for engaging and extracting or removing stones, calculi or other obstructions from the biliary or urinary tracts of a patient are described herein.

### RELATED APPLICATIONS

The present patent document claims the benefit of the filing date under 35 U.S.C. § 119(e) of Provisional U.S. Patent Application Ser. No. 62/518,900, filed Jun. 13, 2017, which is hereby incorporated by reference.

### BACKGROUND

### 1. Technical Field

Methods, medical devices, and kits, and more particularly methods, devices, and kits for engaging and extracting or removing stones, calculi or other obstructions from the biliary or urinary tracts of a patient are described herein.

### 2. Background Information

It is common for various calculi, or “stones,” to form within body passages, such as kidney stones in the ureter or kidneys, and gallstones in bile ducts or the gallbladder. Some stones may be harmless and may pass through the body naturally, for example, gallstones passing through the duodenum and kidney stones through the urethra. However, many other stones may become trapped and may cause serious medical problems, such as abdominal pain, fever, nausea, jaundice, and so forth. If left untreated, calculi can lead to serious health problems (loss of renal function, inflammation) and vitally endanger the patient (sepsis during infected urinary calculus-caused urinary transport disorder). Fast and effective removal of such stones may become necessary.

Endoscopic procedure, such as stone extraction may be performed using baskets, balloons, and/or lithotripsy devices.

An extraction basket may comprise a plurality of wires that deploy in a radially outward direction and are designed to trap the floating stones. However, drawbacks associated with extraction baskets include the need to properly orient the basket, not being able to entrap larger stone fragments in the basket, having smaller stone fragments escape between the basket wires, having the basket not being able to grasp stone fragments of awkward shapes, and having the wires of the basket get caught or snag during removal of the basket with the stones trapped therein.

As an alternative to an extraction basket, a balloon catheter may be inserted through a working lumen of an endoscope to help remove stone fragments. In an exemplary procedure, the balloon is positioned adjacent to and upstream from the stone, inflated, and then moved in a downstream direction to sweep the stone out of the bile duct and into the duodenum. The catheter may comprise multiple lumens for injection of contrast, a wire guide, and inflation of the balloon.

However, in some cases, stones, calculi or the like may be too large (about 2-3 cm in diameter) to be quickly and effectively removed from the tract. The large stones, calculi or the like may either be too big to fit into baskets, or a balloon slips past the stone during attempts to sweep the stone, calculi or the like out of the tract. For example, the stone can be lodged into a pouch near the ampulla of Vater, which often causes the balloon to slip past the stone.

Currently, one option to remove relatively large or trapped stones is to disintegrate a stone into smaller fragments. Although, several procedures are known for disintegrating the stones (e.g., electrohydraulic lithotripsy, laser lithotripsy, ultrasonic lithotripsy, and extracorporeal shock wave lithotripsy (“ESWL”)) and subsequent removal of the smaller stone fragments, disintegrating stones require additional steps contributing to a longer procedure times and less efficient stone removal.

Accordingly, there is a need for an improved stone extraction method and device that is easy to use, effective on large stones, calculi or the like, and reduces the operation time during a stone, calculi or the like removal procedure.

### SUMMARY

One embodiment relates to a device for extracting biliary or urinary stones, calculi or other obstructions from a tract of a patient, the comprising: (i) a two-part gel-forming system comprising: a composition (A) comprising a gel; and a composition (B) comprising one or more crosslinking agents for crosslinking the gel, wherein the two-part gel-forming system is configured to, upon delivery to the tract, conform to the shape of the stones, calculi or other obstructions in the tract and at least partially encapsulate the stones, calculi or other obstructions before the two-part gel-forming system is set in a crosslinked state; and (ii) a delivery system adapted for delivery of the two-part gel forming system, comprising: a catheter shaft; a first expandable apparatus attached to the catheter shaft and being movable between a collapsed position and an expanded position for urging the encapsulated stones, calculi or other obstructions through the entrance and from the tract, wherein the first expandable apparatus is configured to urge stone in a direction along the body passage when in the expanded position; a first longitudinally extending lumen in the catheter shaft in a continuous fluid path with the first expandable apparatus; and, optionally, a second longitudinally extending lumen, wherein, if present, the second longitudinally extending lumen is dimensioned and adapted for delivery of the two-part gel forming system. In some embodiments, the first and second longitudinally extending lumens are in the same catheter shaft. The delivery system may further comprise an additional catheter shaft, and the first and second longitudinally extending lumens are in separate catheter shafts. The second longitudinally extending lumen has an exit port adjacent the first expandable apparatus. The first expandable apparatus may be an inflatable balloon, basket, or braided structure. The device may further comprise a second expandable apparatus, e.g., an inflatable balloon, basket, or braided structure, attached to the catheter shaft and being movable between a collapsed position and an expanded position, the expanded position being adapted for dilating an entrance to the tract and for containing the two-part gel-forming system to a defined length of the tract. The first expandable apparatus may be inflatable to a diameter of about 8 to about 40 mm. The first or the second longitudinally extending lumens may be further adapted for passing of a contrast medium to the tract. The gel may be sodium alginate or carboxymethylcellulose. The one or more crosslinking agents may be multivalent cation salts. The one or more crosslinking agents may be selected from the group consisting of calcium chloride, lithium chloride, magnesium chloride, strontium chloride, barium chloride, aluminum sulfate, barium sulfate, calcium carbonate, ferric chloride, and ferrous chloride. The composition may (B) further comprise a contrast medium. The deliver)/system may further comprise a third lumen extending from a proximal end to a distal end of the delivery system that is dimensioned and adapted for receiving a guide wire therein. The first, second and third longitudinally extending lumens may be in the same catheter shaft or the delivery system further comprises a third catheter shaft, and the first, second, and third longitudinally extending lumens are in separate catheter shafts. The delivery system may comprise four longitudinally extending lumens, and wherein the second lumen is dimensioned and adapted for delivery of the composition (A), and wherein a fourth lumen having an exit port adjacent the first expandable apparatus, the fourth lumen dimensioned and adapted for delivery of the composition (B). The two-part gel-forming system may further comprise a non-steroidal anti-inflammatory agent.

Another embodiment relates to a method for extracting biliary or urinary stones, calculi or other obstructions from a tract of a patient, comprising: (i) positioning a delivery system adjacent to and downstream from a target stone, calculus, or other obstruction, the delivery system comprising: (a) catheter shaft; (b) a first balloon attached to the catheter shaft and being movable between a collapsed position and an expanded position: (c) at least two longitudinally extending lumens, a first lumen for access to the first balloon, a second lumen having an exit port adjacent the first balloon, the second lumen dimensioned and adapted for delivery of a two-part gel forming system, and, optionally a contrast agent: (ii) inflating the first balloon to the expanded position; (iii) delivering through the exit post of the second lumen proximally to the target stone, calculus or other obstruction the two-part gel-forming system in an amount sufficient to at least partially encapsulate the target stone, calculus or other obstructions before the two-part gel-forming system is set in a crosslinked state, the two-part gel-forming system comprising a composition (A) comprising a gel and a composition (B) comprising one or more crosslinking agents for crosslinking the gel. The method may further include (iv) urging the encapsulated stone, calculus or other obstruction through the entrance and from the tract, by retracting the catheter shaft, wherein the first balloon is configured to urge stone in a direction along the body passage when in the inflated state. In the method, the composition (B) is delivered before the composition (A) is delivered. The method may further include (v) passing a buffer through the second lumen between the composition (A) and composition (B) to prevent a premature crosslinking of the two-part gel-forming system within the tract. In the method, the composition (A) and the compositions (B) are delivered simultaneously. In the method, the composition (B) may further comprise a contrast medium. In the method, the catheter shaft may further comprise a second balloon attached to the catheter shaft and being movable between a collapsed position and an expanded position. The method may further comprise (vi) inflating the second balloon to the expanded position to contain the two-part gel-forming system to a defined length of the tract. In the method, the catheter shaft may further comprise a third lumen extending from a proximal end to a distal end of said catheter that is dimensioned and adapted for receiving a guide wire therein.

Yet further embodiment relates to a kit comprising a two-part gel-forming system comprising a composition (A) comprising a gel, and a composition (B) comprising one or more crosslinking agents for crosslinking the gel; and the delivery system described herein. The kit may further comprise a syringe. The delivery system may be a balloon catheter.

### BRIEF DESCRIPTION OF THE DRAWINGS

FIG. 1 is a side view of one embodiment of the described device.

FIG. 2 is a cross-sectional view taken along line 2-2 of FIG. 1.

FIGS. 3, 4, and 5 are partial views of the described device illustrated in FIG. 1.

FIG. 6 is a cross-sectional view of the portions of the device shown in FIG. 1.

FIG. 7 is a cross-sectional view of another embodiment described herein.

FIG. 8 is a photograph of one exemplary described device including a port.

### DETAILED DESCRIPTION

The following detailed description and appended drawings describe and illustrate various exemplary embodiments of the invention. The description and drawings serve to enable one skilled in the art to make and use the invention, and are not intended to limit the scope of the invention in any manner.

Described herein are a two-part cross-linking material (referred to as “two-part gel-forming system” throughout this application) and devices, kits, and methods that deliver the two-part cross-linking material into a tract or a duct, e.g., bile duct, pancreatic duct, ureter, or kidneys to assist in removing large (larger than 2 cm in diameter, typically 2-3 cm in diameter) gallstones, pancreatic stones, or kidney stones, respectively.

The first part (i.e., “composition (A)”) of the two-part cross-linking material includes a material to be cross-linked, e.g., gel or a fluid having viscosity that is higher than a cross-linker; the second part (i.e., “composition (B)”) is a low-viscosity solution, which is the cross-linker. The two parts can be injected in either order or simultaneously.

As used herein the terms “comprise(s),” “include(s),” “having,” “has,” “contain(s),” and variants thereof, are intended to be open-ended transitional phrases, terms, or words that do not preclude the possibility of additional acts or structure. The use of “e.g.,” “etc.,” “for instance,” “in example,” and “or” and grammatically related terms indicate non-exclusive alternatives without limitation, unless otherwise noted. The use of “optionally” and grammatically related terms means that the subsequently described element, event, feature, or circumstance may or may not be present or occur, and that the description includes instances where said element, event, feature, or circumstance occurs and instances where it does not. The use of “attached” refers to the fixed, releasable, or integrated association of two or more elements and/or devices. Thus, the term “attached” includes releasably attaching or fixedly attaching two or more elements and/or devices. As used herein, the terms “proximal” and “distal” are used to describe opposing axial ends of the particular element or feature being described. The terms “tract” or “duct” can be used interchangeably throughout the instant application and refer to any lumen within the body of an animal, including, but not limited to, humans, and includes elongate passages. As examples, the term includes ducts, such as bile duct, pancreatic duct, ureter, or ducts in the kidneys. The term “animal” includes human and other mammals. The methods, medical devices and kits described herein are particularly well-suited for use in tracts and ducts but can indeed be used in any suitable body vessel.

The device for extracting biliary or urinary stones, calculi or other obstructions from a tract of a patient includes: (i) a two-part gel-forming system comprising a composition (A) comprising a gel and a composition (B) comprising one or more crosslinking agents (also referred to as “crosslinkers”) for crosslinking the gel, wherein the two-part gel-forming system is configured to, upon delivery to the tract, conform to the shape of the stones, calculi or other obstructions in the tract and at least partially encapsulate the stones, calculi or other obstructions before the two-part gel-forming system is set in a crosslinked state; and (ii) a delivery system dimensioned and adapted for delivery of the two-part gel-forming system that includes a catheter shaft comprising a first expandable apparatus (such as a balloon, basket, or braided structure) attached to the catheter shaft and being movable between a collapsed position and an expanded position for urging the encapsulated stones, calculi or other obstructions through the entrance and from the tract, wherein the first expandable apparatus is configured to urge stone in a direction along the body passage when hi the expanded position, and a first longitudinally extending lumen in the catheter shaft in continuous fluid path with the first expandable apparatus, and optionally, a second longitudinally extending lumen, wherein, if present, the second longitudinally lumen is dimensioned and adapted for delivery of the two-part gel-forming system.

In certain embodiments where the delivery system includes both, the first and the second longitudinally extending lumens, the first and second longitudinally extending lumens may be located in the same catheter shaft. Alternatively, the delivery system may further comprise an additional catheter shaft (i.e., second catheter shaft), and the first and second longitudinally extending lumens are located in separate catheter shafts. The first and the second longitudinally extending lumens may have an exit port adjacent the first expandable apparatus.

Optionally, the delivery system may also include a third lumen extending from a proximal end to a distal end of said catheter that is dimensioned and adapted for receiving a guide wire therein. The third lumen may be located in the same catheter shaft as the first and the second lumens or may be located in a separate catheter shaft (i.e., a third catheter shaft).

Compositions (A) and (B) can be introduced one after the other or together, whereby it is preferred that composition (B) is introduced before composition (A) to guarantee a suitable distribution and complete embedding of all of the target stone(s) before start of and during crosslinking, respectively. The formed gels exhibit sufficient stability and flexibility to encapsulate the stone(s) and be extracted together with the stones by sweeping the stone out of the duct.

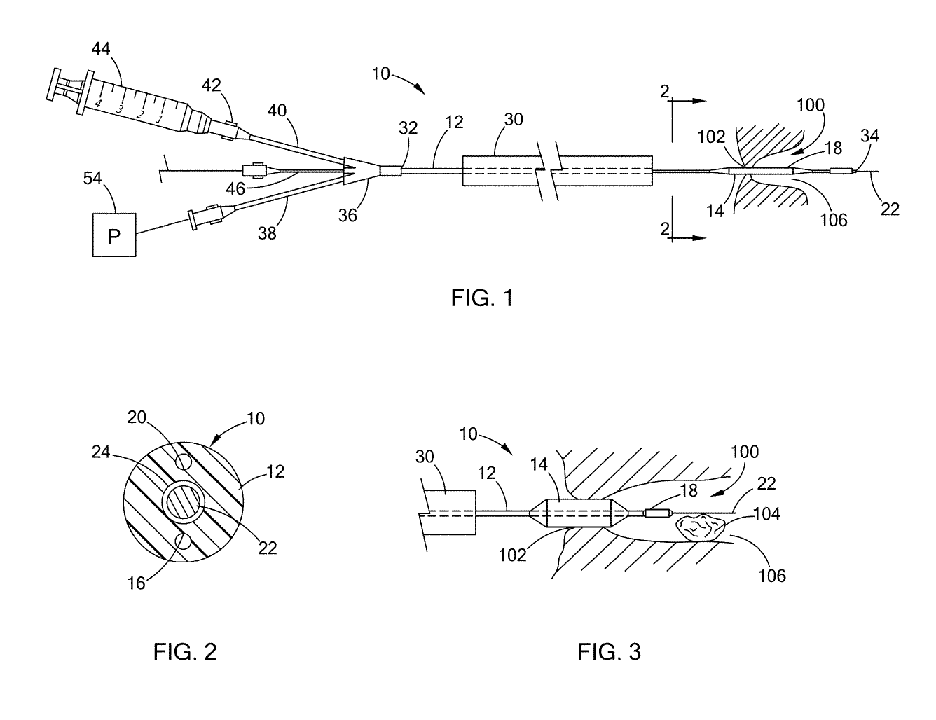

With reference first to FIGS. 1 to 3, an embodiment of a delivery system, a device 10 described herein is there shown, useful for delivery of the two-part gel-forming system and extracting biliary or urinary stones, calculi or the like 104 (hereinafter, stone 104) from the biliary or urinary tract 100 of a patient. The device 10 and the other embodiments of the present invention are most preferably employed for stone extraction from the biliary tract 100, and will therefore be described with respect to a stone 104 located within the hepatopancreatic duct 106 (also known as “ampulla of Vater”). In certain embodiments, the device 10 may also be employed for a temporary dilation of the Sphincter of Oddi 102 within the biliary tract 100.

The device 10 comprises a catheter shaft 12 having a proximal end 32 and a distal end 34. In certain embodiments, the catheter shaft 12 comprises silicone or another suitable medical grade material. When intended for use at the ampulla of Valter, the catheter shaft 12 may be about 5 to about 7 French (1.67 to 2.33 mm) in size. Moreover, as explained in more detail below, when the device 10 is employed for engaging the stone 104 via an antegrade approach (i.e., the device 10 is introduced into the patient through the patient's mouth), the catheter shaft 12 may be about 180 to about 200 cm long.

The device 10 further comprises a first inflatable balloon 18 attached/fixed on the catheter shaft 12. The first balloon 18 is dimensioned and adapted for urging the stone 104 through the biliary or urinary tract 100 of the patient, when in an expanded or inflated state. Preferably, the first balloon 18 is dimensioned and adapted for urging the stone 104 through the Sphincter of Oddi 102 and into the small intestine or duodenum for passing (the Sphincter of Oddi 102 can be temporally dilated to its dilated diameter by inflation of an optional, second expandable apparatus, such as a second balloon 14 and deflation of the second balloon 14, and before closure of the Sphincter of Oddi 102 to its undilated diameter). If both balloons are present, the first balloon 18 is preferably affixed or attached to the catheter shaft 12 distal of the second balloon 14. The first balloon 18 advantageously comprises latex or another suitable medical grade material of comparable softness or elasticity such as silicone rubber. The selection of such other material for the first balloon 18 should be well within the skill of the art, for example, on a trial-and-error basis after considering softness or elasticity.

The first balloon 18 is preferably inflatable to a diameter about 2 to about 5 mm larger than the inflated diameter of the first balloon. A particularly preferred shape for the first balloon 18 is shown in cross-section in FIG. 6. The first balloon 18 is advantageously inflatable to a diameter of about 8 to about 40 mm, and to a volume of about 1.5 to about 4 cm³. It highly desirable that the device described herein be arranged to affirmatively establish a tension in the first balloon, which is suitable for urging movement of the stones, calculi or the like encapsulated in the two-part gel-forming system (i.e., crosslinked gel), and minimize any risk of rupture of the first balloon from overpressure. For this purpose, the device 10 of the present invention preferably also comprises a structure 44 (FIG. 1) for delivery of a fixed, predetermined volume of inflation medium to the first balloon 18. The structure 44 can comprise the syringe shown in FIG. 1 or another suitable structure, such as the Wilson-Cook Q.B.I.D.™ apparatus.

As noted above, in certain alternative embodiments, the device 10 may also comprise a second inflatable balloon 14 fixed or attached to the catheter shaft 12. The second inflatable balloon 14 is preferably dimensioned to be identical in size as the first balloon 18 (i.e., inflatable to a diameter of about 8 to about 40 mm, and to a volume of about 1.5 to about 4 cm³) and, also, adapted for trapping the two-part gel-forming system described herein from spreading, e.g., downstream in the duct to unintentional locations following the delivery of the two-part gel-forming system, and, in some embodiments, for the temporary dilation of a tract entrance, such as the Sphincter of Oddi, from an undilated diameter (FIG. 1) to a dilated diameter (FIG. 3), in a manner described herein. To this end, the second balloon 14 is preferably inflatable to a diameter of about 8 to about 40 mm, and to a volume of about 1.5 to about 4 cm³. The second balloon 14 preferably comprises a compliant material, e.g., latex, silicon, or urethane material.

In an embodiment with a first balloon 18 only, the catheter shaft 12 of the device 10 includes one, two, or three longitudinally extending lumens 16, 20 and 24 defined therein (FIG. 2). Additional longitudinally extending lumens (not shown) may also be present.

The first longitudinally extending lumen 16 is dimensioned and adapted for the supply of a suitable inflation medium to the first balloon 18. The first lumen 16 is connected via a coupling 36 to an inlet 38 for the inflation medium, for example, supplied from a pressurized source 54 of the inflation medium. In an instance where only one lumen is present in the catheter shaft, the first lumen 16 may also be dimensioned and adapted for delivery of the two-part gel forming system, and, optionally a contrast agent or medium (e.g., a contrast dye). For example, the first lumen 16 may also include an exit port adjacent to the first balloon 18 for the delivery of the two-part gel-forming system and a contrast agent. Specifically, in one embodiment, a contrast agent can be mixed with the cross-linker to minimize procedure steps and volume added to the bile duct. This also allows where the cross-linker is traveling within the biliary tree, which is preferred.

It is noted that any suitable visualization agent that facilitates visualization of the location of an injection can be used in this step, and contrast dye is merely an example. A skilled artisan will be able to select a suitable visualization agent for use in a method according to a particular example based on various considerations, including the nature and location of the body vessel for which modification of the luminal profile is desired and the nature of any visualization devices and/or equipment being used during performance of the method. Other examples of suitable visualization agents include all agents visible under fluoroscopy and all agents visible under ultrasound.

In an embodiment with two lumens, the first and the second lumens may be located in the same catheter shaft or be located in separate catheter shafts. The optional, second lumen 20 may be similarly or differently dimensioned, having an exit port 28 (FIG. 6) adjacent the first balloon 18 and being dimensioned and adapted for delivery of a two-part gel forming system described herein, and, optionally, a contrast agent or medium (e.g., a contrast dye) through the exit port 28 and into the biliary or urinary tract 100 of the patient. The exit port 28 is shown proximal of the first balloon 18 in FIG. 6. The second lumen 20 is connected via the coupling 36 to an inlet 40, connected in turn to the delivery structure 44.

The optional third longitudinally extending lumen 24 is dimensioned and adapted for receiving a guide wire 22 therein, shown here to be located in the same catheter shaft as the first and the second lumens. In an alternative embodiment, the delivery system may include a third catheter shaft dimensioned and adapted for receiving a guide wire (not shown). The guide wire 22 can be a conventional 0.89 mm (0.035 in.) guide wire, or other guide wire suitable for the intended use of the device 10. The device 10 can further comprise such a guide wire 22 receivable in the third lumen 24.

Imaging during use of the device 10 can be carried out in several ways. It is an important advantage of the present invention, however, that the stone 104 and the engagement of the first balloon 18 with the stone 104 can be viewed directly through the temporarily dilated Sphincter of Oddi 102. To this end, the device 10 is introduced into the patient via an endoscope, such as an over-the-wire endoscope 30 (shown only schematically), to which the catheter shaft 12 is connectable. Such connection occurs, for example, by feeding the catheter shaft 12 through the endoscope 30. The device 10 can of course further comprise the endoscope 30.

Alternatively, depending upon the circumstances of use, it may be desirable to image the device 10 or the stone 104 by means of radioscopy, fluoroscopy or the like. Such circumstances may arise, for example, if the stone 104 is located some distance into the duct. The device 10 can further comprise at least one radiopaque band 48 or 50 (and preferably a pair of radiopaque bands 48 and 50) positioned on the catheter shaft 12 immediately proximal or distal of the first balloon. The radiopaque bands 48 and 50 can comprise a suitable metal or other medical grade material, and are shown in FIGS. 6 and 7.

In another embodiment, depending upon the circumstances of use, it may be desirable to image the device 10 or the stone 104 using a contrast agent that may be delivered via the second lumen 20.

In alternative embodiment, similarly for imaging, the device 10 can also or alternatively comprise a fourth longitudinally extending lumen 26 defined in the catheter shaft 12 (see FIGS. 6 and 7), also having an exit port 108 also adjacent the first balloon 18 and being dimensioned and adapted for a separate delivery of a contrast medium through the exit port 108 and into the biliary or urinary tract 100 of the patient. The exit port 108 is shown proximal of the first balloon 18 in FIG. 6, and distal of the first balloon 18 in FIG. 7. Alternatively, should the need for a contrast medium arise, it is also possible to instead either inject the contrast medium around the guide wire 22, or remove the guide wire 22 from the third lumen 24 and introduce the contrast medium through the third lumen 24.

In all methods, medical devices and kits, a suitable gel (i.e., composition (A)) can be used that is paired with one or more crosslinking agents (i.e., composition (B)). Examples of suitable types of cross-linkable materials include liquids, gels, foams, fluidized biomaterials, and other materials. Examples of gels include sodium alginate or carboxymethyl cellulose. The inventors have determined that viscous liquids having an injection viscosity of between about 50,000 and about 200,000 cP in a cannula ranging between about 23 to about 27 gauge are considered particularly well-suited for injecting a crosslinkable gel in the inventive methods, medical devices, and kits, particularly when being used to aid in removal of the stones from the urinary, pancreatic, and biliary tracts. Carboxymethyl cellulose hydrogel and alginate is considered particularly well-suited for use as a gel material in the inventive methods, medical devices and kits. A hydrogel containing between about 4% alginate and about 30% alginate is considered particularly advantageous for use as a gel for ability to inject and ability to crosslink. Indeed, the inventors have determined that a hydrogel containing about 17.5% alginate is particularly advantageous for use as a gel to be crosslinked to aid in the removal of stones.

In all methods, medical devices and kits, any gel in composition (A) can be used that is paired with a composition (B) that includes one or more crosslinking agents. Addition of a crosslinking agent can allow the gel to undergo a phase change and/or increase the gel's viscosity above its injection viscosity. Crosslinking of the gel could occur via electrostatic or covalent crosslinking. Example electrostatic crosslinkers include but are not limited to ligand-receptor interactions, such as streptavidin-biotin interactions, or ionic interactions from molecules containing divalent and trivalent elements, such as MgCl₂, CaCl₂, SrCl₂, BaCl₂, Al₃SO₄, BaSO₄, CaCO₃, ferric chloride, and ferrous chloride. Another example includes LiCI.

The inventors have determined that SrCl₂ is particularly well-suited for use as a crosslinking agent in the methods, devices and kits described herein. The inventors have determined that the inclusion of SrCl₂ at a concentration of between about 0.05 Molar and about 4.00 Molar is particularly advantageous. Indeed, the inventors have determined that the inclusion of SrCl₂ at a concentration of about 0.33 Molar is particularly advantageous. Covalent crosslinking could be achieved by inclusion of a free radical generator, click chemistry, Schiff base reactions, and enzyme-mediated reactions. Additionally, stimuli responsive gels can be used that contain component(s) that trigger the crosslinking process, for example by light activation (added photoinitiator), temperature activation, or pH activation (added acid or base). It is considered advantageous for the crosslinked gel to have a compressive modulus of 10 to 3000 kPa, preferably 100 to 500 kPa.

When a crosslinking agent and the gel are mixed together, interaction between the crosslinking agent and the gel can occur before or after the gel is delivered to the target location. When the gel and crosslinking agent interact before they are delivered to the target location, it is considered advantageous to have delayed crosslinking to facilitate delivery of the gel. If delayed crosslinking is used, it is advantageous to maintain an injection viscosity of between about 1 and about 1,000,000 cP for the time required to deliver the combined gel and crosslinking agent to the target location. When the gel and crosslinking agent interact after they are delivered to the target location (i.e., in-situ crosslinking) the gel and crosslinking agent can be delivered with a single or separate delivery that occurs simultaneously or sequentially. If single delivery is used, a dual beveled needle with a dual lumen catheter can be used to separate the gel and crosslinking agent in the medical device used for delivery. If separate delivery of the gel and crosslinking agent is used, the same delivery device can deliver each agent sequentially or each agent can be delivered with a separate delivery device simultaneously or sequentially. If sequential delivery of the gel agent and crosslinking agent is performed, a suitable buffer may be used to add a layer that separates the agents within the delivery lumen of the cannula. If included, the buffer may be a gel that does not interact with the crosslinker, a suitable liquid, such as saline, phosphate saline buffer, or water, or any other suitable buffer.

The inventors have determined that the viscosity ranges listed above provide effective viscosities for use in stone extraction procedure described herein.

In certain embodiment, the composition B of the two-part gel-forming system described herein may also include a contrast medium.

In certain further embodiments, the two-part gel-forming system may include therapeutic agents. Therapeutic agents may be delivered simultaneously with, before or after the composition (A) and/or composition (B) of the two part gel-forming system are delivered adjacent to and downstream from the target stone. One example of therapeutic agents that may be delivered include, but are not limited to, anti-inflammatory agents, such as non-steroidal anti-inflammatory drugs (NSAIDs).

Use of the device 10 and the two-part gel-forming system of the present invention for extracting the stone 104 from the patient can now be easily understood.

The procedure is intended to be performed under a combination of endoscopic and fluoroscopic imaging, known as endoscopic retrograde cholangiopancreatography (ERCP).

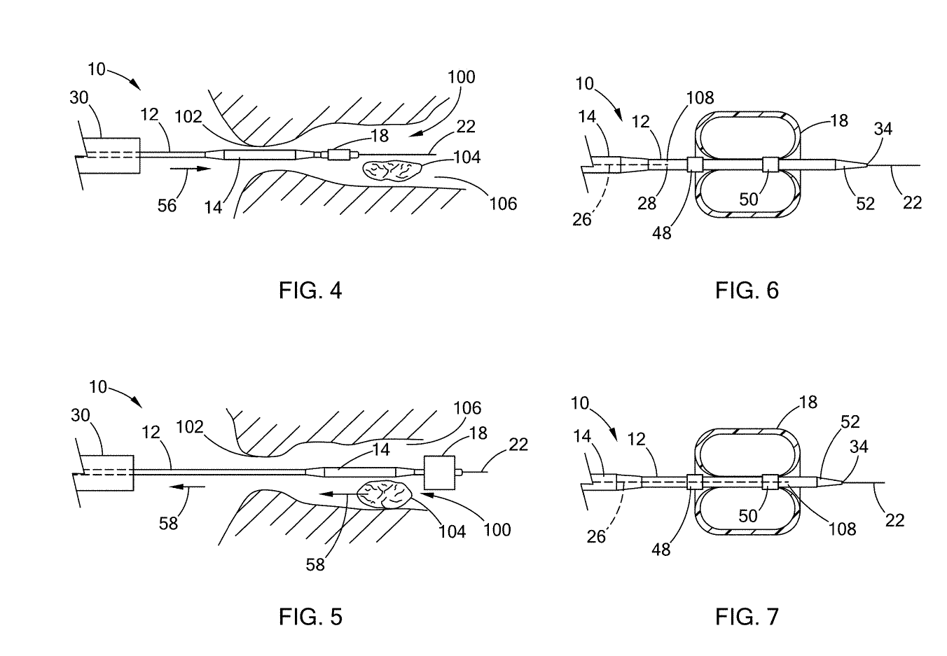

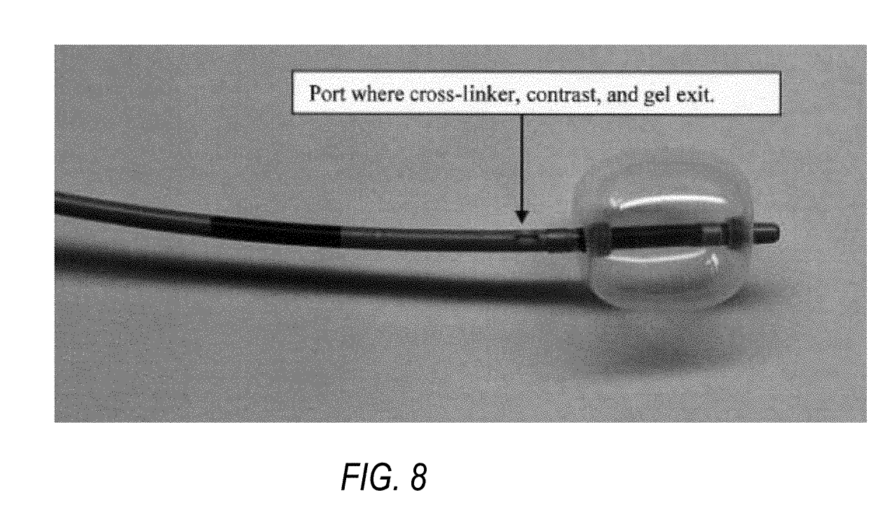

As mentioned above, the relatively small profile of the delivery system, device 10, readily allows the catheter shaft 12 and the deflated first balloon 18 (and, optionally, second balloon 14) to be advanced along a previously inserted guide wire 22, for example, extending through the Sphincter of Oddi 102 and into the duct. Such a position for the guide wire 22 and device 10 is shown in FIG. 1, the endoscope 30 first having been introduced into the patient via an antegrade approach and advanced along the guide wire 22. In an embodiment, where the second balloon is used, once the second balloon 14 lies fully across the Sphincter of Oddi 102, the second balloon 14 may be inflated so as to dilate the Sphincter of Oddi 102 (FIG. 3). The second balloon 14 is then deflated so as to allow observation of the first balloon 18 and the stone 104 through the Sphincter of Oddi 102 via the endoscope 30 (FIG. 4). The device 10 is advanced in the direction of arrow 56 until the first balloon 18 has passed the stone 104 and is positioned adjacent to and downstream from a target stone, calculus or other obstruction 104. The first balloon 18 is then inflated (FIG. 5).

Once the first balloon 18 is fully inflated, the two-part gel-forming system comprising a composition (A) comprising a gel, and a composition (B) comprising one or more crosslinking agents for crosslinking the gel is injected via the first lumen 16, or if the second lumen 20 is present, via the second lumen 20 into the biliary or urinary tract 100 of the patient. The composition (A) and composition (B) may be injected in either order or injected simultaneously. Preferably, composition (B) is injected before compositions (A) is injected. The composition (A) and composition (B) are injected via a skive or a port 28 (FIG. 6.) in the catheter that is proximal to the first balloon 18 until the cross-linking agent(s) surrounds the target stone 18. The skive can be proximal or distal to the target stone 18. The crosslinking agents and crosslinkable gels are fluids, which can conform to the shape of the stone 104 so that the stone is at least partially or completely encapsulated before setting in the crosslinked state (i.e., to form a “plug”). In addition, the crosslinked gel fills the inner diameter of the biliary or urinary tract 100 of the patient, at least partially, but preferably the gel will completely fill the tract, so that the first balloon will not slip past the crosslinked gel when the stone is removed. The first balloon 18 will have more surface area in contact with the plug, which is attached to the stone 104, therefore making it easier to remove the stone 104 and less likely to slip by the stone. In certain embodiments, the catheter shaft 12 of the device 10 can include yet another longitudinally extending lumen to allow for delivery of the composition (A) and composition (B) simultaneous through different longitudinal lumens. This device design advantageously allows the delivery of the two-part gel-forming system with the same catheter without having to perform an exchange.

In one embodiment, the composition (B) in injected first. The delivery lumen may then be purged with a small volume of a buffer (e.g., saline; about 1 mL) to prevent premature crosslinking within the lumen of the catheter. The composition (A) is then injected into the tract to achieve a cross-linked gel around the stone.

In certain embodiments, the cross-linking agents and/or the gels can be mixed with a contrast medium so that the user can track how far into the tract or ducts the injection travels. This is to prevent the injections from traveling too far beyond the desired location of the stone and to prevent a cross-linked gel from setting in an area that would be difficult to remove by endoscopic techniques. Preferably, one or the other of the gel/cross-linker combination would have contrast so that they can be distinguished from one another, unless they are injected simultaneously in which case they will mix together.

Once the two-part gel-forming system has substantially crosslinked, the first balloon 18 is manipulated to urge the stone 104, which is encased in the two-part gel-forming system, which has set, in the direction of arrow 58 through the Sphincter of Oddi 102 and into the duodenum of the patient. The device 10, the endoscope 30 and the guide wire 22 are then withdrawn from the patient.

Of course, the stone 104 can be manipulated by other structures, which may be useful for extracting biliary or urinary stones, calculi or the like (such as stone 104) from the biliary or urinary tract 100 of a patient. Such other, exemplary structures were previously described in U.S. Pat. No. 6,692,484, which is incorporated herein by reference in its entirety.

The device 10 and the two-part gel-forming system provides for both the engagement or extraction and removal of large stones, calculi or the like and the temporary dilation of a structure in the biliary or urinary tract 100, preferably of the Sphincter of Oddi 102, without requiring removal of the dilation balloon and introduction of another balloon or basket. This significantly shortens the time necessary for the successful performance of the extraction or removal procedure, while avoiding the need to surgically cut the Sphincter of Oddi or other structure in the patient. Without regard to whether the device 10 or some prior art apparatus is employed, however, it is important that the two-part gel-forming system is delivered and at least partially or completely encapsulates the stone 104.

In an alternative embodiment, where the second balloon 14 is used, the cross-linked gel can be formed around the stone without the gel spreading downstream in the tract/duct to unintentional locations. This keeps the gel contained to a defined length of duct so that it does not travel to upper side branches in the biliary tree, the pancreatic duct (if there are no pancreatic stones), or the cystic duct.

The proposed device is advantageous over previously described stone extraction devices: the surgeon would be able to proceed to the extraction of a large stone that has been at least partially, or preferably, completely encapsulated by the described two-part gel-forming system that, also, completely fills the inner diameter of the biliary or urinary tract of the patient so that the balloon will not slip past the stone during the stone extraction or removal process.

In view of the foregoing, it should be clear that the device 10 is provided with a two-part gel-forming system for dilating sphincters or other tract entrances in the body, and engaging and extracting or removing stones, calculi or the like from the biliary or urinary tract of a patient. The devices of the present invention may permit efficient and fast engagement and extraction or removal of stones, calculi or the like from the biliary tract, the urinary tract or other body structure without requiring any surgical cutting of body tissue.

The details of the construction or composition of the various elements of the device 10 not otherwise disclosed are not believed to be critical to the achievement of the advantages of the present invention, so long as the elements possess the strength or mechanical properties needed for them to perform as disclosed. The selection of any such details of construction are believed to be well within the ability of one of even rudimentary skills in this area, in view of the present disclosure. For practical reasons, however, the device 10 should probably be considered to be single-use devices, rather than being reusable.

Yet, further embodiments relate to a kit for extracting biliary or urinary stones, calculi or other obstructions from a tract of a patient. In addition to the components described throughout this application in connection with a medical device, the kit may also include a syringe, balloon catheter, and gel delivery catheter; in some embodiments, the gel and balloon catheters may be the same catheters.

### INDUSTRIAL APPLICABILITY

The present invention is useful for promptly removing stones, calculi, or other obstructions from the body of a human or veterinary patient, for example, from the biliary or urinary tract of the patient, and therefore finds applicability in human and veterinary medicine.

It is to be understood, however, that the above-described device is merely an illustrative embodiment of the principles of this invention, and that other devices and methods for using them may be devised by those skilled in the art, without departing from the spirit and scope of the invention. It is also to be understood that the invention is directed to embodiments both comprising and consisting of the disclosed parts.

1. A device for extracting biliary or urinary stones, calculi or other obstructions from a tract of a patient, the comprising:
(i) a two-part gel-forming system comprising:
a) a composition (A) comprising a gel; and
b) a composition (B) comprising one or more crosslinking agents for crosslinking the gel,
wherein the two-part gel-forming system is configured to, upon delivery to the tract, conform to the shape of the stones, calculi or other obstructions in the tract and at least partially encapsulate the stones, calculi or other obstructions before the two-part gel-forming system is set in a crosslinked state; and
(ii) a delivery system adapted for delivery of the two-part gel forming system, comprising:
a) a catheter shaft;
b) a first expandable apparatus attached to the catheter shaft and being movable between a collapsed position and an expanded position for urging the encapsulated stones, calculi or other obstructions through the entrance and from the tract, wherein the first expandable apparatus is configured to urge stone in a direction along the body passage when in the expanded position;
c) a first longitudinally extending lumen in the catheter shaft in a continuous fluid path with the first expandable apparatus; and
d) optionally, a second longitudinally extending lumen;
wherein, if present, the second longitudinally extending lumen is dimensioned and adapted for delivery of the two-part gel forming system.

2. The device of claim 1, wherein the first and second longitudinally extending lumens are in the same catheter shaft.

3. The device of claim 1, wherein the delivery system further comprises an additional catheter shaft, and wherein the first and second longitudinally extending lumens are in separate catheter shafts.

4. The device of claim 1, wherein the second longitudinally extending lumen has an exit port adjacent the first expandable apparatus.

5. The device of claim 1, wherein the first expandable apparatus is an inflatable balloon, basket, or braided structure.

6. The device of claim 1, further comprising a second expandable apparatus attached to the catheter shaft and being movable between a collapsed position and an expanded position, the expanded position being adapted for dilating an entrance to the tract and for containing the two-part gel-forming system to a defined length of the tract, the second expandable apparatus being an inflatable balloon, basket, or a braided structure.

7. The device of claim 1, wherein the first expandable apparatus is inflatable to a diameter of about 8 to about 40 mm.

8. The device of claim 1, wherein the first or the second longitudinally extending lumens are further adapted for passing of a contrast medium to the tract.

9. The device of claim 1, wherein the gel is sodium alginate or carboxymethyl cellulose.

10. The device of claim 1, wherein the one or more crosslinking agents are multivalent cation salts.

11. The device of claim 10, wherein the one or more crosslinking agents are selected from the group consisting of calcium chloride, lithium chloride, magnesium chloride, strontium chloride, barium chloride, aluminum sulfate, barium sulfate, calcium carbonate, ferric chloride, and ferrous chloride,

12. The device of claim 1, wherein the composition (B) further comprises a contrast medium.

13. The device of claim 1, wherein the delivery system further comprises a third lumen extending from a proximal end to a distal end of the delivery system that is dimensioned and adapted for receiving a guide wire therein.

14. The device of claim 13, wherein the first, second and third longitudinally extending lumens are in the same catheter shaft.

15. The device of claim 13, wherein the delivery system further comprises a third catheter shaft, and the first, second, and third longitudinally extending lumens are in separate catheter shafts.

16. The device of claim 1, wherein the delivery system comprises at least four longitudinally extending lumens, and wherein the second lumen is dimensioned and adapted for delivery of the composition (A), and wherein a fourth lumen having an exit port adjacent the first expandable apparatus, the fourth en dimensioned and adapted for delivery of the composition (B).

17. The device of claim 1, wherein the two-part gel-forming system further comprises a non-steroidal anti-inflammatory agent.

18. A method for extracting biliary or urinary stones, calculi or other obstructions from a tract of a patient, comprising:
(i) positioning a delivery system adjacent to and downstream from a target stone, calculus, or other obstruction, the delivery system comprising:
a) catheter shaft;
b) a first balloon attached to the catheter shaft and being movable between a collapsed position and an expanded position;
c) at least two longitudinally extending lumens, a first lumen for access to the first balloon, a second lumen having an exit port adjacent the first balloon, the second lumen dimensioned and adapted for delivery of a two-part gel forming system, and, optionally a contrast agent;
(ii) inflating the first balloon to the expanded position;
(iii) delivering through the exit post of the second lumen proximally to the target stone, calculus or other obstruction the two-part gel-forming system in an amount sufficient to at least partially encapsulate the target stone, calculus or other obstructions before the two-part gel-forming system is set in a crosslinked state, the two-part gel-forming system comprising:
a) a composition (A) comprising a gel; and
b) a composition (B) comprising one or more crosslinking agents for crosslinking the gel.

19. The method of claim 18, further comprises (iv) urging the encapsulated stone, calculus or other obstruction through the entrance and from the tract, by retracting the catheter shaft, wherein the first balloon is configured to urge stone in a direction along the body passage when M the inflated state.

20. A kit comprising:
(i) a two-part gel-forming system comprising:
a) a composition (A) comprising a gel, and
b) a composition (B) comprising one or more crosslinking agents for crosslinking the gel;
(ii) a delivery device adapted for delivery of the two-part gel forming system, comprising:
a) a catheter shaft;
b) a first expandable apparatus being movable between a collapsed position and an expanded position, wherein the first expandable apparatus is configured to urge stones, calculi or other obstructions in a direction along the body passage when in the expanded position, and
c) a first longitudinally extending lumen in the catheter shaft in a continuous fluid path with the first expandable apparatus.
